(19) United States
(12) Patent Application Publication
(10) Pub. No.: US 2026/0020784 A1
(43) Pub. Date: Jan. 22, 2026

(54) MONITORING METABOLIC RESPONSE

(71) Applicant: GLUCOSE REPUBLIC LTD, Cardiff (GB)

(72) Inventors: Julian Douglas Llewellyn SHAPLEY, Cardiff (GB); David Joseph HAINES, Cardiff (GB)

(21) Appl. No.: 19/341,730

(22) Filed: Sep. 26, 2025

Related U.S. Application Data
(63) Continuation of application No. PCT/GB2024/050730, filed on Mar. 18, 2024.

(30) Foreign Application Priority Data
Mar. 29, 2023 (GB) 2304634.5

(51) Int. Cl. A61B 5/145; A61B 5/00

(52) U.S. Cl. CPC A61B 5/14532; A61B 5/4836; A61B 5/7292; A61B 5/7435

(57) ABSTRACT

Simulating a user's metabolic system and predicting a blood glucose response based on a specified food intake event includes receiving characteristic data representative of specified user characteristics, converting said characteristic data into object data and utilising said object data and a master model to generate a personal digital model representative of said user's metabolic system, receiving food intake data representative of a said specified food intake event, obtaining or generating current glucose data for said user, inputting said food intake data to said personal digital model, and generating, using said personal digital model, a simulation of said user's metabolic system in response to said food intake data and generating a predicted blood glucose response to said specified food intake event, outputting data representative of said predicted blood glucose response, and utilising said data representative of said predicted blood glucose response to retrain said master model and said personal digital model.

### FIELD OF THE INVENTION

This invention relates generally to monitoring the metabolic response of a user and, more particularly, to a computer-implemented method and system for generating, using physiological data received in respect of a specified user, a digital metabolic system that simulates that specified user's metabolic system so as to enable one or more parameters or characteristics of the user's blood glucose levels to be monitored, determined or predicted.

### BACKGROUND OF THE INVENTION

Blood glucose monitors exist, that can be used to test the concentration of glucose in a user's blood. Particularly important in diabetes management, a blood glucose test is typically performed by piercing the skin to draw blood, then applying the blood to a chemically active disposable ‘test-strip’. The other main option is continuous glucose monitoring. Different manufacturers use different technology, but most systems measure an electrical characteristic and use this to determine the glucose level in the blood. Skin-prick methods measure capillary blood glucose, whereas CGM correlates interstitial fluid glucose level to blood glucose level. Measurements may occur after fasting or at random non-fasting intervals, each of which informs diagnosis or monitoring in different ways.

All of these methods of blood glucose measurement only enable blood glucose management to be performed in a reactive way. In other words, if the user's blood sugar is too high at the instant of testing, the user can take action to rectify that and, in the same way, if their blood sugar is too low at the instant of testing, the user can take rectifying action. Also, this type of testing is typically only performed by users that already have a condition like diabetes that requires careful management, whereas most people that do not have such a condition, tend not to monitor their blood sugar levels.

However, it is well documented that managing blood sugar levels is important for the overall health and well being of all people, as well as for the prevention and/or management of conditions such as diabetes, and it would be advantageous to provide a method and system that enables a user to carefully control their blood glucose levels in a proactive, rather than reactive, manner, and without the need for regular blood glucose testing.

Aspects of the present invention seek to address at least one or more of these issues.

### SUMMARY OF THE INVENTION

In accordance with a first aspect of the invention, there is provided a computer-implemented method for simulating a specified user's metabolic system and predicting a blood glucose response for that user in respect of a specified food intake event, the method includes, under control of a processor, the steps of receiving user characteristic data representative of specified user characteristics, converting said characteristic data into object data and utilising said object data and a master model to generate a personal digital model representative of said user's metabolic system, receiving food intake data representative of a said specified food intake event, obtaining or generating current glucose data for said user, inputting said food intake data to said personal digital model, and generating, using said personal digital model, a simulation of said user's metabolic system in response to said food intake data and thereby generating a predicted blood glucose response to said specified food intake event, outputting data representative of said predicted blood glucose response, and utilising said data representative of said predicted blood glucose response to retrain said master model and said personal digital model.

In an exemplary embodiment, the step of generating a predicted glucose response may comprise generating glucose excursion prediction data utilising said personal digital model.

Beneficially, the step of generating glucose excursion prediction data may include generating a predicted glucose excursion for a specified period of time following a said specified food intake event. Optionally, the method may further comprise generating, using said personal digital model and said glucose excursion prediction data, a meal score representative of said user's glucose response to a said food intake event.

In an exemplary embodiment, the method may further comprise generating, using said predicted blood glucose response and said personal digital model, recommendation data representative of recommended alternative foods that would improve the user's blood glucose response to a said food intake event, and outputting said recommendation data.

In accordance with another aspect of the invention, there is provided a computer-implemented system for simulating a specified user's metabolic system and predicting a blood glucose response for that user in respect of a specified food intake event, the system including a processor, a memory in which is stored a set of executable instructions, and a user interface provided on an app which is downloadable onto a user's computing device, the system being configured to, under control of the processor, to execute said executable instructions to receive, via said user interface, user characteristic data representative of specified user characteristics, convert said characteristic data into object data and utilising said object data and a master model to generate a personal digital model representative of said user's metabolic system, receive, via said user interface, food intake data representative of a said specified food intake event, obtain or generating current glucose data for said user, input said food intake data to said personal digital model, and generate, using said personal digital model, a simulation of said user's metabolic system in response to said food intake data and thereby generate a predicted blood glucose response to said specified food intake event, output, via said user interface, data representative of said predicted blood glucose response, and utilise said data representative of said predicted blood glucose response to retrain said master model and said personal digital model.

Once again, the step of generating a predicted glucose response may comprise generating glucose excursion prediction data utilising said personal digital model.

Optionally, the step of generating glucose excursion prediction data may include generating a predicted glucose excursion for a specified period of time following a said specified food intake event.

In an exemplary embodiment, the system may be configured to generate, using said personal digital model and said glucose excursion prediction data, a meal score representative of said user's glucose response to a said food intake event.

The system may be further configured to generate, using said predicted blood glucose response and said personal digital model, recommendation data representative of recommended alternative foods that would improve the user's blood glucose response to a said food intake event, and output said recommendation data via said user interface.

In accordance with another aspect of the invention, there is provided a downloadable app configured to provide, on a user's computing device, a user interface for use with the computer-implemented system substantially as described above.

In accordance with yet another aspect of the invention, there is provided a computer program product including instructions for implementing the method substantially as described above.

These and other aspects of the invention will be apparent from the following detailed description.

### BRIEF DESCRIPTION OF THE DRAWINGS

Embodiments of the present invention will now be described, by way of examples only, and with reference to the accompanying drawings, in which;

FIG. 1 is a schematic block diagram illustrating an example system for simulating a user's metabolic system;

FIG. 2 is a schematic block diagram illustrating an example personalised system for simulating a user's metabolic system;

FIG. 3 is a schematic flow diagram illustrating an example method for simulating a user's metabolic system;

FIG. 4 is an extract, illustrated in tabular form, of an example data frame used to train an example system for simulating a user's metabolic system;

FIG. 5 is a schematic block diagram of an example process for deriving a glucose model;

FIG. 6 is a schematic block diagram illustrating an example system for simulating a user's metabolic system;

FIG. 7 is a sequence flow diagram illustrating schematically an example system for simulating a user's metabolic system;

FIG. 8 is a schematic block diagram illustrating an example system for simulating a user's metabolic system; and

FIG. 9 is a sequence flow diagram illustrating an example method for simulating a user's metabolic system.

### DETAILED DESCRIPTION OF THE INVENTION

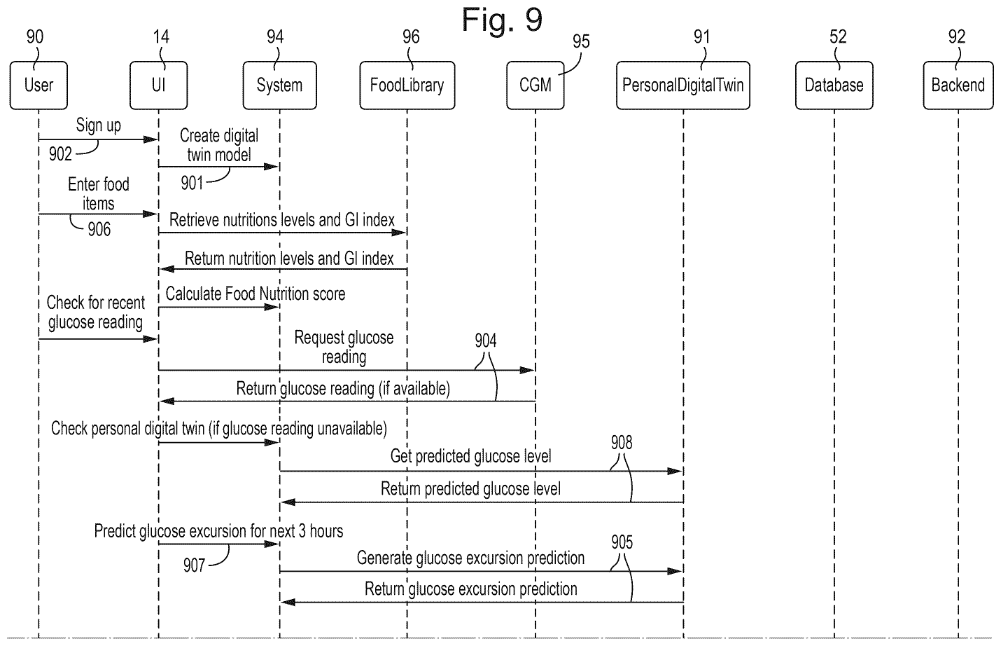

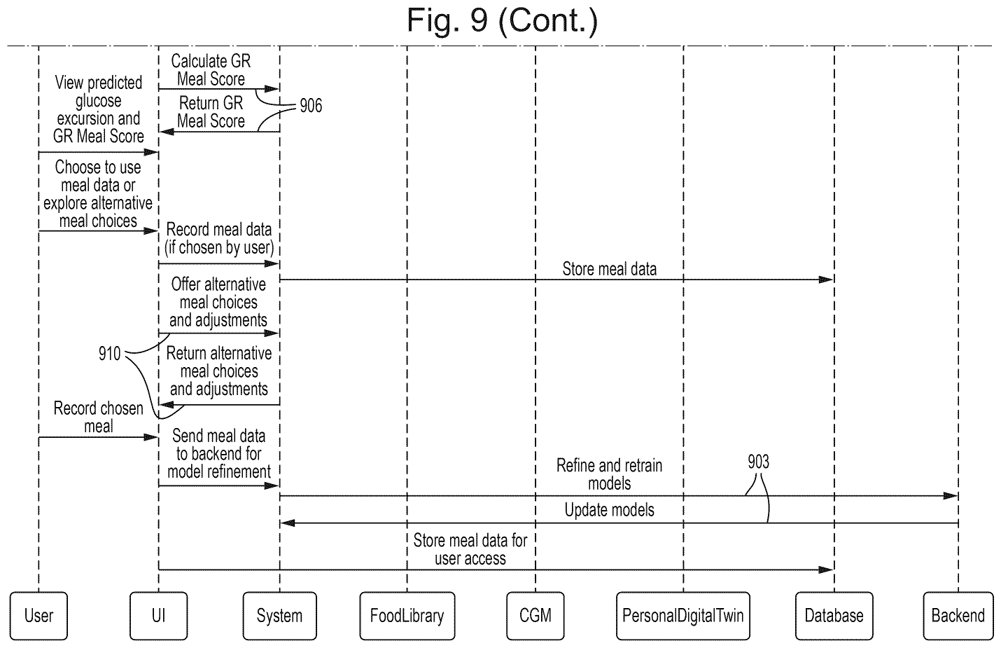

In a computer-implemented system and method according to an exemplary aspect of the invention, and as illustrated schematically in the sequence flow diagram of FIG. 9, a personalised digital model 91 is generated, at step 901, in respect of a specified user 90, that simulates that user's metabolic system. The personalised digital model (or digital ‘metabolic twin 91’) is derived (at the backend 92 of the system) from a master ML model trained using training data derived from a number of trial users. The training data comprises personal characteristic data representative of the trial users and their blood glucose readings over time and in response to various foods consumed. Other data, such as activity level, etc. may also be derived from the trial users and used to train the master ML model. When a user 90 first accesses the system via a user interface (UI) 14 provided on a wellness app, they will enter their personal characteristic data (during a sign up step 902), and they will be assigned a personal ML model 91 based on their physiological profile, such that it acts, in use, to simulate the user's metabolic system in response to data representative of food intake, and can, for example, be used to predict the effect of a specific food or combination of foods, if consumed, on a user's blood glucose levels, both instantaneously and over a subsequent time period, and/or to predict future blood glucose levels or specific blood glucose ‘events’, e.g. spikes. In use, real blood glucose measurements obtained from the specified user is used to retrain and refine their personal ML model and also to retrain and refine the master ML model (both represented by steps 903), such that the app for each user will become more and more accurate over time, as will the master ML model.

In an exemplary system, a Glucose Model is used to implement various features of the app, such as generating a personalised food score representative of the effect on the user's blood glucose levels of a particular meal, and even providing suggestions for changes that could be made to the meal to improve the food score. Other features and advantages will be apparent from the following description.

### The Master ML Model

A digital ‘metabolic twin’ referenced herein is a digital model that can be used to simulate the metabolic system of a specified user. This digital model can be used in an example system of the invention, and in response to input data representative of food (or combinations of food) consumed (or to be consumed) by that user, predict the effect on the user's blood glucose level by consuming that food (or combination of food). The digital model uses a number of key parameters to capture essential variables that will affect a user's glucose response to specified food intake. An embodiment of an example system starts with a master machine learning (ML) model implemented in, for example, the Python programming environment, that defines the column headers (parameters) for the ML model and outlines the process of training the model using, for example, a Pandas library, which will be familiar to a person skilled in the art of programming in programming languages such as Python. The blood glucose levels recorded after each meal (or food intake) are used as the target variable for the ML model, and other data points (such as personal user data, meal data, and time (of day) data) are used as features to train the ML model. Once the master ML model is trained, it can be used to create personalised digital ‘metabolic twins’ for individual specified users, using personalised user data to refine the digital model (metabolic twin) and its predictions.

Initially, a number of parameters (column headers) are defined for the ML model. These may, for example, comprise any or all of the following;
1. Glucose level: This is the target variable that the model will predict. It represents the user's glucose level after a meal, which is the model's primary focus.
2. Time of day: This parameter represents the time of day when the glucose level was recorded. It can be useful in capturing any diurnal variations in glucose levels.
3. Sex: This parameter represents the sex of the user, which can impact their glucose response to meals.
4. Age: This parameter represents the age of the user, which can also impact their glucose response to meals.
5. Weight: This parameter represents the weight of the user, which can impact their glucose response to meals.
6. Height: This parameter represents the height of the user, which can impact their glucose response to meals.
7. Meal content: This parameter represents the content of the meal, including the macro and micronutrient levels. This parameter can be split into multiple sub-parameters, such as carbohydrate, protein, fat, and fibre content.
8. Meal time: This parameter represents the time of the meal, which can impact the glucose response.
9. Baseline glucose level: This parameter represents the user's glucose level before the meal, which can impact the glucose response.

The ML model is configured to transform these parameters into object data defining the column headers. Thus, in an example system, the master ML model may define objects or ‘classes’ for ‘Person’, ‘Meal’ and ‘GlucoseReading’. The Person object may be defined using personalised data, such as sex, weight, height and age. The Meal object may be defined using meal content and meal time. The GlucoseReading object may be defined using a time stamp (meal time), glucose level (actual or predicted), the Person class, the Meal class, and the time of the last meal.

Thus, the Person class represents information about a person, such as their sex, weight, height, and age.

The Meal class represents information about a meal, such as its carbohydrate, protein, and fat content, as well as the time at which it was consumed.

The GlucoseReading class represents a single glucose reading, along with information about the person who took the reading, the meal they consumed, and the time that has elapsed since the meal was consumed. The time_since_meal attribute is calculated based on the time of the current reading and the time of the most recent previous reading with the same meal time.

The process of training the Master ML model is initialised, in this example, by importing a data analysis library (i.e. Pandas, in this example) into the programming environment, and then creating a list for each of the Person objects, the Meal objects, and also the Time objects (which are simply specified times of day). A dictionary is also created for holding Glucose Prediction data, such as:
sex, age, weight, and height;
meal data, e.g. carbohydrate content, protein content, fat content, fibre content, and also meal time;
time of day; and
a baseline glucose level.

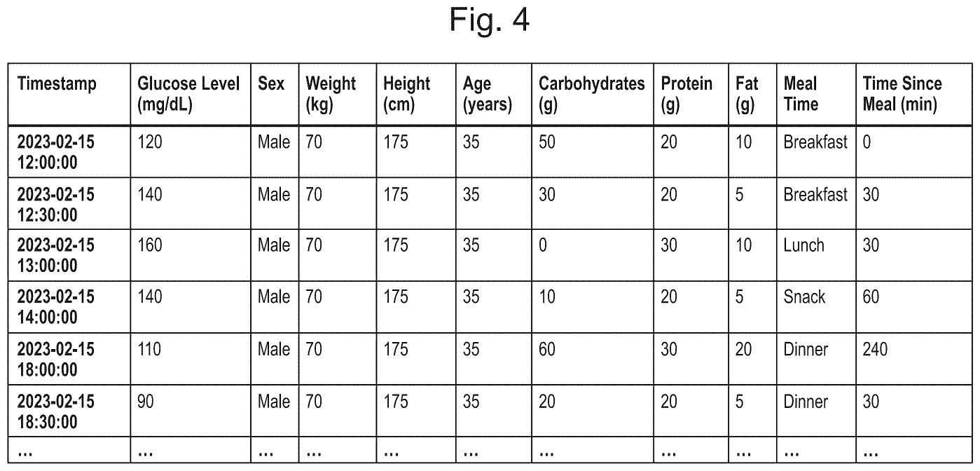

The Person, Meal and Time objects are used to create lists of data combined into a dictionary, in which the ‘keys’ are the column headers and the values are lists of corresponding data points. A final dataframe is then constructed with the dictionary as an argument, wherein the dataframe includes columns for all of the relevant data points, as defined in the Glucose Prediction data referenced above. A simplified illustration of a dataframe used for training the master ML model is illustrated in FIG. 4 of the drawings.

Once the dataframe has been constructed, it can be used to train the model, wherein the glucose levels recorded after each meal are used as a target variable for the model, and the other data points (Person, Meal and Time) are used as features to train the model.

The above provides a simplified example of a Glucose Model that can be used to train the master ML model. In a more realistic example, the Person class might additionally include an activity value, which could be represented numerically or as a categorical variable (e.g. sedentary, moderate, active), and a fasting glucose level measured after an overnight fast, which can provide additional information about a person's glucose metabolism. Also, the Meal class may include additional data points in respect of the composition of a meal, such as one or more of sugar, sodium, potassium, calcium, iron, vitamins a, c, d, cholesterol, saturated fat and unsaturated fat.

Additional models are beneficially combined with he resultant master ML model to derive a Glucose Model, as referenced hereinafter. Such additional models might include ‘Area Under the Curve’ (AUC) and a food score for glucose excursions for a particular meal and a particular user, as described hereinafter.

When the master ML model has been trained, it can be used in an example system to create personalised models (or digital ‘metabolic twins’) for individual users. As will be described in more detail hereinafter, in order to do this, a new row in the dataframe is created and the model is retrained using this new data point. This personalised ML model (or digital ‘metabolic twin’) can then be used to predict a respective individual user's glucose response to a specific meal.

### The Glucose Models

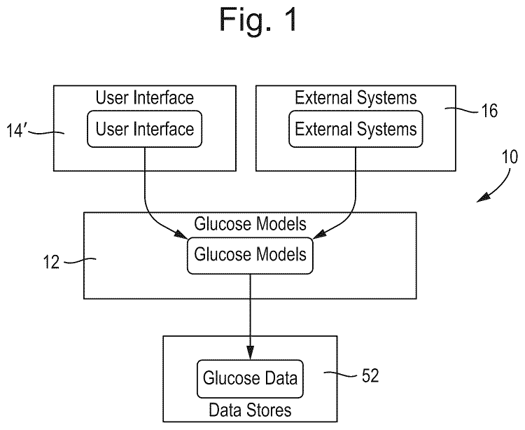

Referring to FIG. 1 of the drawings, once the master ML model has been trained using real Glucose Prediction data derived from a trial involving, say, 50+ people, it can be deployed in an example system 10, that allows an individual user to enter their personal data and meal information via a user interface 14′ (received from, for example, a wellness app), and this personal data and meal information can then be used by the Glucose Model module 12 to generate glucose data which is stored in a data store 52. External systems 16 may be called or accessed by the Glucose Model module 12 to obtain additional information, such as the composition of a specific food entered by the user, for example.

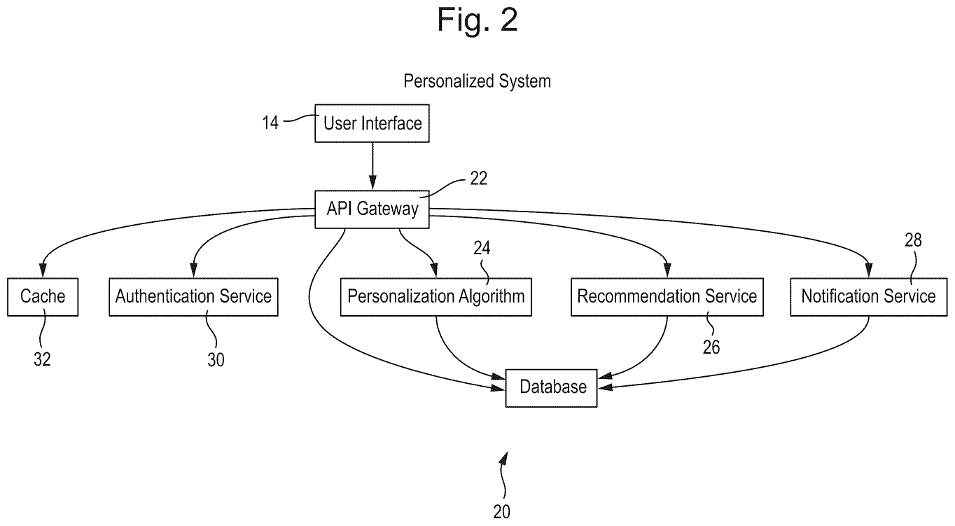

Referring to FIG. 2 of the drawings, a wellness app 20 may be provided, comprising a user interface 14 and an API gateway 22 that provides the interface and gateway between the app 20 and a glucose prediction APIs provided in an example system. The wellness app 20 also comprises a personalisation module 24, a recommendation module 26 and a notification module 28. An authentication module 30 and a cache 32 are also provided, as is fairly standard in many apps.

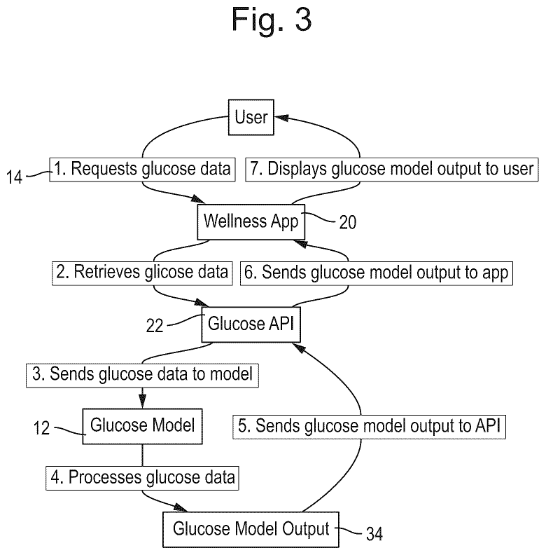

Referring additionally to FIG. 3 and also to FIG. 9 of the drawings, the wellness app 20 can request and receive glucose data from the user (at steps 904), via the user interface 14, and transmit the glucose data to the glucose models 12 (via the API gateway 22), where it is compared with predicted glucose data to generate a Glucose model output 34. The Glucose model output data is collected (and stored), and used to retrain the Master ML model and the user's personal model (i.e. their digital ‘metabolic twin’ 91).

### The Glucose Model

As referenced above, additional models are beneficially combined with the Master ML model to derive he Glucose model 12. Such models might include AUC and a food score for glucose excursions for a particular meal and specified user.

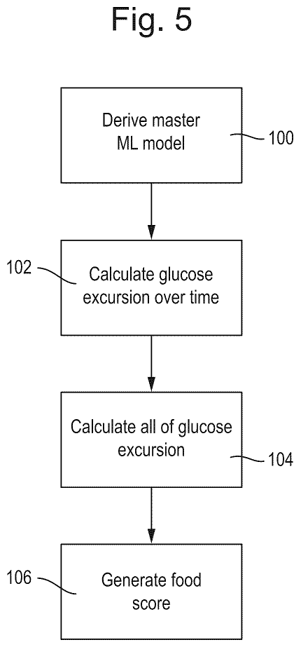

Referring to FIG. 5 of the drawings, in a process for deriving a Glucose model 12 for use in an example system for monitoring metabolic response, at step 100, the Master ML model is derived, as described above. At step 102, a function is defined to calculate glucose excursion over time for a given meal and user, using the Master ML model and the personalised digital ‘metabolic twin’. This may take the form of/;
a. using the Master ML model to simulate a typical metabolic system (according to the user's personal characteristics) to predict glucose levels for the given meal;
b. using the personalised digital ‘metabolic twin’ to simulate the user's metabolic system and adjust the glucose predictions based on user data;
c. calculating a glucose excursion (e.g. at 5 minute intervals) over a given time period (e.g. 3 hours) using the adjusted glucose prediction and a user baseline glucose value.

The metabolic system simulations for a. and b. above may be achieved using a regression process, for example extra trees regression, wherein each decision tree is constructed using a random subset of features and a random subset of training samples. After constructing a large number of decision trees, the algorithm takes the average of the predictions made by each tree to produce a final prediction. As a result, the model can be made much more robust and accurate than would be possible using, for example, a single decision tree, and this method helps to reduce overfitting and increases the generalisation power of the model. Indee, extra tree regression has significant advantages over other learning algorithms, including the ability to handle noisy data and outliers well. Also, it enables a faster training time due to the randomisation of feature and sample selection (rather than training using the whole training dataset every time). The algorithm is trained on many samples, as discussed in relation to the Master ML model above, and then the glucose excursion prediction can be achieved by running the algorithm as a for loop over the selected time period for each time point.

At step 104, a function is defined (in the system 94, FIG. 9) to calculate the AUC of the glucose excursion over the given time period. In order to achieve this, a numerical library, such as NumPy (in Python, as will be familiar to a person skilled in the art) can be imported into the programming environment and used to calculate the AUC of glucose excursion over the selected time period, using the time point values (e.g. at 5-minute intervals over the selected time period) and calculated glucose excursion values derived using the calculated glucose excursion applied at each of the time points (at steps 905, FIG. 9).

At step 106, these functions can be combined to create a function that takes, as inputs, a meal and specified user (at step 906—FIG. 9), and returns, not just data representative of the glucose excursion over the selected time period (if required), but also a personalised score for that meal based on the AUC of the glucose excursion. This (combined) function determines the glucose excursion, as described above, calculates the AUC, as described above, and then converts the AUC into a score (e.g. on a scale of 1 to 10).

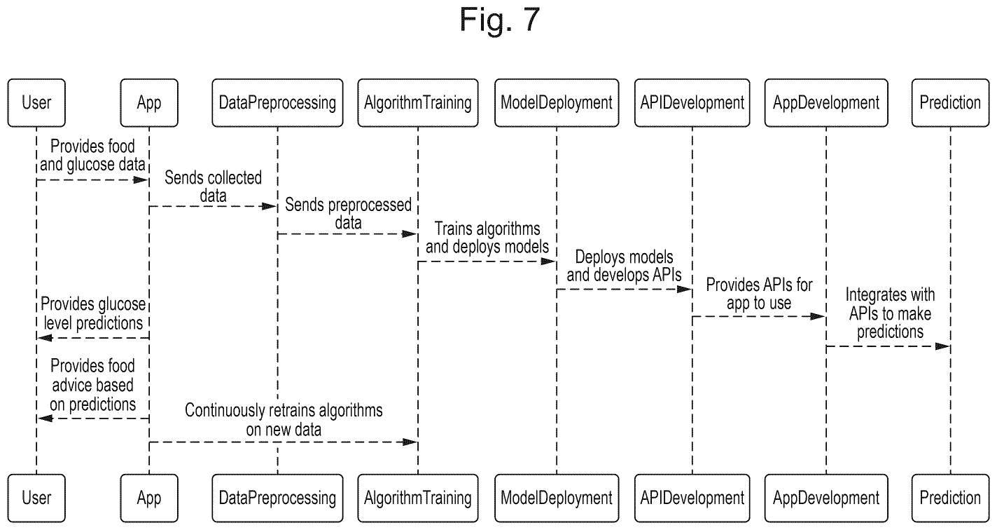

Thus, to summarise and referring to FIG. 7 of the drawings, in an example system, an exemplary process may be performed, comprising the steps of:
1. The user provides food and glucose data to the app.
2. The app sends the collected data to DataPreprocessing
3. DataPreprocessing sends the preprocessed data to Algorithm Training
4. Algorithm Training trains the algorithms and deploys the models
5. The models are deployed, and APIs are developed in APIDevelopment
6. APIDevelopment provides APIs for the app to use
7. The app integrates with the APIs to make predictions and provide glucose level predictions to the user.
8. The app also provides food advice based on the predictions
9. The algorithms are continuously retrained (e.g. using extra tree regression) on new data in Algorithm Training.

Note that the metabolic digital twin is used in the AlgorithmTraining to simulate the user's metabolic system and make predictions based on historical data. If a user is using a continuous glucose monitor (CGM) (95—FIG. 9), the comparison may, alternatively, be made between the predicted glucose level and the actual CGM readings. If no CGM is available, the food-based prediction is made based on the metabolic digital twin's predicted levels.

The above description provides the basis for generating and deploying both the Master ML model, the personalised ML model (i.e. an individual user's digital ‘metabolic twin’) in terms of that user's glucose response to various foods and combinations of foods, and a Glucose model for predicting a glucose response to an entered meal, for example, and providing glucose excursion data and/or a food score as described above. Multiple users can access and utilise an example system of the invention via a wellness app 20 running on various respective personal computing devices. When a user first creates an account on their app, they will be prompted to enter personal characteristic data (such as age, sex, weight, height and activity level). These characteristics will be used, by the system, to generate a digital ‘metabolic twin’ for that user, which is derived from the trained Master ML model using the input personal characteristic data. The user can then input meal data and the system then uses that meal data, first to generate glucose excursion data using the Master ML model, then to adjust that glucose excursion data by utilising the meal data in a simulation of the user's metabolic system using their personal digital ‘metabolic twin’, and finally to calculate the AUC of the adjusted glucose excursion to generate a food score, as described above. Any real blood glucose data received (from a continuous glucose monitor, for example) in respect of that user can be fed back to the system and used to retrain and refine both the Master ML model and the user's personal digital ‘metabolic twin’ (9-FIG. 9), as described in more detail below with reference to FIG. 9 of the drawings.

Additionally, in an exemplary system, a novel method of classifying foods and meals (as described hereinafter) may be deployed to generate personal food scores for the user, and even to provide recommendations to improve that user's food score. Additionally, or alternatively, in an exemplary system, a novel method of predicting glucose spikes (as described hereinafter) may be deployed, that predicts, not just the glucose excursion over the selected time period, but also identifies potential glucose spikes.

### The Food Score and Recommendation Module

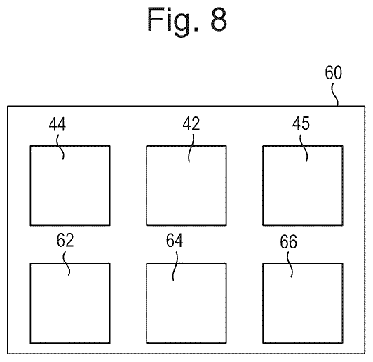

Referring to FIG. 8 of the drawings, the Food Score and Recommendation modules 42, 44 may be incorporated in a deep learning clustering system 60 that is configured to classify foods and meals based on their impact on blood sugar levels. Thus, the deep learning clustering system includes the food score module 42 and the recommendation module 44, and also a data input module 62 for receiving data representative of a meal, a deep learning clustering module 64 for analysing the input data to determine the impact of each ingredient in the input meal on blood sugar levels and a classification module 66 for grouping the ingredients into different categories

Once the user has configured their app and a personalised digital model (91—FIG. 9) that acts, in use, to simulate their metabolic system has been generated and deployed, the user can enter data representative of foods and meals (at step 906—FIG. 9). A novel deep learning clustering module is utilised in an example system, the deep learning clustering module being configured to classify foods and meals (using the food library 96—FIG. 9) based on their impact on blood sugar levels. Data representative of the ingredients of a meal can be entered via the user interface 14 provided by the wellness app on a user's personal computing device, and that food data is transmitted to the system, via the API.

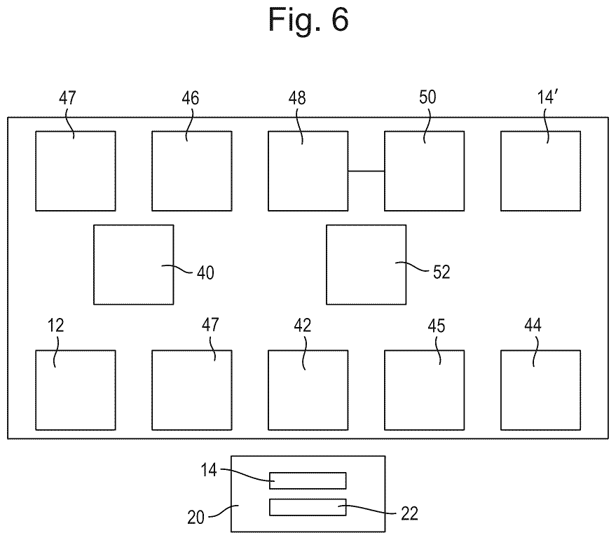

Thus, referring to FIG. 6 of the drawings, an example system 10 for simulating a user's metabolic system includes a deep learning clustering module 40 for grouping the ingredients into different categories based on their impact on blood sugar. The system may also include a food score module 42 for generating an overall score for the meal based on the combined impact of all of the ingredients on blood sugar levels (step 106, FIG. 5, FIG. 9), and, optionally, a recommendation module 44. The system may comprise a prediction module 45 for predicting blood glucose levels over the next predetermined time period (e.g. three hours) (at step 907, FIG. 9) based on data about the person's food intake, physical activity, and other relevant factors.

The system may also include a pre-planning module for assisting users in planning their meals based on the scores and recommendations of the system. The deep learning clustering system disclosed herein is useful for helping people make better choices about what to eat and manage their blood sugar levels and for supporting weight loss, overall health, and metabolic health.

The deep learning clustering module 60 incorporates a food clustering algorithm, a prediction algorithm, a person clustering algorithm and a food advice algorithm. The personalised digital model for use in simulating a specified user's metabolic system (i.e. their digital ‘metabolic twin’ 91—FIG. 9), which is generated when the user configures their app (as described above), uses the food clustering, prediction and person clustering algorithms as input, and each digital ‘metabolic twin’ is trained using supervised learning techniques, such as the extra tree regression as referenced above, as will be familiar to a person skilled in the art. Each such digital ‘metabolic twin’ will, thus, be able to accurately simulate the user's metabolic system and provide highly accurate and personalised predictions for each respective user, and that accuracy and personalisation will increase and improve over time, as the Master ML model and personalised model are retrained and refined.

To summarise, briefly, the principal modules utilised in an example system:

### Food Clustering Algorithm

The food clustering algorithm is responsible for grouping foods into clusters based on their macro and micronutrient content. The algorithm will be trained on a comprehensive food library and will use unsupervised learning techniques such as k-means clustering or hierarchical clustering to group foods into clusters.

### Prediction Algorithm

The prediction algorithm is trained on user glucose level data, recorded along with the food intake data. The algorithm uses supervised learning techniques such as linear regression, decision trees, or neural networks to make predictions based on the food intake data. The predictions generated by the algorithm will be refined over time as the algorithm is retrained on new user data.

### Person Clustering Algorithm

The person clustering algorithm is responsible for grouping users into clusters based on their food intake and glucose level patterns. The algorithm uses unsupervised learning techniques such as k-means clustering or hierarchical clustering to group users into clusters.

### Metabolic Digital Twins

The metabolic digital twins are personalised models trained on specific users. These models use the food clustering, prediction, and person clustering algorithms as input and are trained using supervised learning techniques such as linear regression, decision trees, or neural networks. The metabolic digital twins will provide highly accurate and personalised predictions for each user.

### Food Advice Algorithm

The food advice algorithm provides users with recommendations for food choices based on the predictions made by the system. The algorithm uses the predictions as input, and incorporates information about the food clustering and person clustering algorithms to provide users with personalised food advice. This can be utilised by the pre-planning module referenced above, and/or to offer alternative meal choices and/or adjustments to a chosen meal (step 910—FIG. 9) to improve the user's glucose response (GR).

The algorithms and models used in the system can be continuously retrained on new data as it is generated by users, thus facilitating improved accuracy and maintained relevance over time. A monitoring and maintenance module (FIGS. 6-47) is configured to retrain all of the above algorithms and models as required.

In an exemplary method, when a user first configures the wellness app (20—FIG. 2), they are first asked to input (via the user interface 14) their personal characteristics, such as gender, age, height and weight. This data is stored in a database (e.g. 52—FIG. 6), and the user's digital metabolic twin is generated and stored, as described above. The user can then enter food information (step 906, FIG. 9) via the user interface (14—FIG. 2, FIG. 9), and the food clustering algorithm is configured to use a food library (96—FIG. 9) to group food into clusters based on their macro and micronutrient content, as described above. Next, the prediction algorithm is called, which is configured to predict the user's glucose response to the input food, if consumed (step 907—FIG. 9). This glucose prediction may take the form of a predicted blood glucose excursion over a subsequent period of time (e.g. 3 hours) based on a current blood glucose level (which could be a real blood glucose measurement if the user has a CGM (95—FIG. 9) and, otherwise, based on the predicted current blood glucose level (step 908—FIG. 9) derived from using the digital metabolic twin (91—FIG. 9) to simulate the user's metabolic system. Next, the person clustering algorithm is called, and the output from that is utilised in the personalisation algorithm (24—FIG. 2) that utilises the user's digital metabolic twin and the above-referenced food advice algorithm which, as described above, outputs personalised food advice based on the food input data and the outputs from the person clustering algorithm and the prediction algorithm. This advice is displayed to the user, via the user interface (14—FIG. 2, FIG. 9).

The food input data, glucose prediction and personalised advice is stored in a database (e.g. 52—FIG. 6, FIG. 9) and the monitoring and maintenance module (47—FIG. 6) is configured to retrain the food clustering algorithm, the prediction algorithms, the personalisation algorithm, the digital metabolic twin and the food advice algorithm, as well as itself, using newly generated and stored data (which can then form part of the training dataset for use in, for example, an extra tree regression process.

### Glucose Spike Prediction

In an example system, as explained above, the user's digital metabolic twin (i.e. personalised ML model that simulates that user's metabolic system) and deep learning clustering are used to predict blood glucose levels for the next selected period of time (e.g. 3 hours) (glucose excursion) in response to food data input and based on a current blood glucose level which may be a real reading from a cgm or a predicted blood glucose value derived from previous simulations of the user's metabolic system using their digital metabolic twin. In a further extension of this aspect, the prediction algorithm may be configured to predict, from the glucose excursion prediction data, potential glucose spikes based on data representative of the user's food intake, physical activity and other relevant factors. Once again, and if a potential spike is identified by the prediction algorithm, the food advice algorithm may be configured to provide recommendations to the user to avoid or minimise such spikes. These recommendations may, for example, include eating larger or smaller portions of food, eating an alternative food type and/or increasing/decreasing physical activity.

As well as the various modules referred to in the description above (and, optionally, others, not so described), the system 10 will also typically comprise an MCU/MPU device 46 including a number of individual components including, but not limited to, one or more microprocessors 47, a memory 48 (e.g. volatile memory such as RAM) for the loading of executable instructions 50 defining the functionality the MCU/MPU 46 carries out under control of the processor 47. A data store 52, e.g. a database, is also provided. It will be understood that although the various modules and devices included in the system 10 are illustrated in FIG. 6 in a single unit, they may be provided separately and remote from each other. At least some of the modules may advantageously be implemented virtually, via the Cloud for example, and the present invention is not intended to be limited in this regard.

It will be apparent to a person skilled in the art, from the foregoing description, that modifications and variations can be made to the described embodiments without departing from the scope of the invention as defined in the appended claims.

1. A computer-implemented method for simulating a specified user's metabolic system and predicting a blood glucose response for that user in respect of a specified food intake event, the method comprising, under control of a processor, the steps of:
receiving user characteristic data representative of specified user characteristics;
converting said characteristic data into object data and utilising said object data and a master model to generate a personal digital model representative of said user's metabolic system;
receiving food intake data representative of a said specified food intake event;
obtaining or generating current glucose data for said user;
inputting said food intake data to said personal digital model, and generating, using said personal digital model, a simulation of said user's metabolic system in response to said food intake data and thereby generating a predicted blood glucose response to said specified food intake event;
outputting data representative of said predicted blood glucose response; and
utilising said data representative of said predicted blood glucose response to retrain said master model and said personal digital model.

2. A computer-implemented method according to claim 1, wherein the step of generating a predicted glucose response comprises generating glucose excursion prediction data utilising said personal digital model.

3. A computer-implemented method according to claim 1, wherein said step of generating glucose excursion prediction data includes generating a predicted glucose excursion for a specified period of time following a said specified food intake event.

4. A computer-implemented method according to claim 2, comprising generating, using said personal digital model and said glucose excursion prediction data, a meal score representative of said user's glucose response to a said food intake event.

5. A computer-implemented method according to claim 1, further comprising generating, using said predicted blood glucose response and said personal digital model, recommendation data representative of recommended alternative foods that would improve the user's blood glucose response to a said food intake event, and outputting said recommendation data.

6. A computer-implemented system for simulating a specified user's metabolic system and predicting a blood glucose response for that user in respect of a specified food intake event, the system comprising a processor, a memory in which is stored a set of executable instructions, and a user interface provided on an app which is downloadable onto a user's computing device, the system being configured to, under control of the processor, to execute said executable instructions to:
receive, via said user interface, user characteristic data representative of specified user characteristics;
convert said characteristic data into object data and utilising said object data and a master model to generate a personal digital model representative of said user's metabolic system;
receive, via said user interface, food intake data representative of a said specified food intake event;
obtain or generating current glucose data for said user;
input said food intake data to said personal digital model, and generate, using said personal digital model, a simulation of said user's metabolic system in response to said food intake data and thereby generate a predicted blood glucose response to said specified food intake event;
output, via said user interface, data representative of said predicted blood glucose response; and
utilise said data representative of said predicted blood glucose response to retrain said master model and said personal digital model.

7. A computer-implemented system according to claim 6, wherein the step of generating a predicted glucose response comprises generating glucose excursion prediction data utilising said personal digital model.

8. A computer-implemented method according to claim 6, wherein said step of generating glucose excursion prediction data includes generating a predicted glucose excursion for a specified period of time following a said specified food intake event.

9. A computer-implemented method according to claim 7, comprising generating, using said personal digital model and said glucose excursion prediction data, a meal score representative of said user's glucose response to a said food intake event.

10. A computer-implemented method according to claim 6, wherein the system is further configured to generate, using said predicted blood glucose response and said personal digital model, recommendation data representative of recommended alternative foods that would improve the user's blood glucose response to a said food intake event, and output said recommendation data via said user interface.

11. A downloadable app configured to provide, on a user's computing device, a user interface for use with the computer-implemented system according to claim 6.

12. A computer program product comprising instructions for implementing the method of claim 1.
